(19) United States
(12) Patent Application Publication
(10) Pub. No.: US 2026/0295301 A1
(43) Pub. Date: Oct. 1, 2026

(54) METHODS OF USING A COMBINATION OF ALTERNATING ELECTRIC FIELDS, RADIATION THERAPY, TEMOZOLOMIDE AND CHECKPOINT INHIBITORS

(71) Applicant: NOVOCURE GMBH, Root D4 (CH)

(72) Inventors: Moshe Giladi, Haifa (IL); Uri Weinberg, Haifa (IL); Maya Sadovnik, Haifa (IL)

(21) Appl. No.: 19/578,297

(22) Filed: Mar. 25, 2026

Related U.S. Application Data
(60) Provisional application No. 63/777,400, filed on Mar. 25, 2025.

(51) Int. Cl. A61N 5/10; A61K 31/495; A61K 39/00; C07K 16/28

(52) U.S. Cl. CPC A61N 5/1048; A61K 31/495; C07K 16/2818; C07K 16/2827; A61K 2039/545

(57) ABSTRACT

Disclosed are methods of treating a subject having glioblastoma comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject; and wherein the maintenance phase comprises applying an alternating electric field to a target site of the subject for a period of time, wherein the target site comprises one or more glioblastoma cells; administering a therapeutically effective amount of TMZ; and administering a therapeutically effective amount of a checkpoint inhibitor to the subject, wherein the maintenance phase occurs after the treatment phase and after stopping radiation therapy.

### CROSS-REFERENCE TO RELATED APPLICATIONS

This application claims the benefit of U.S. Provisional Patent Application No. 63/777,400, filed on Mar. 25, 2025, which is incorporated by reference herein in its entirety.

### BACKGROUND

Glioblastoma is an aggressive cancer bearing a very poor prognosis. With the best treatment regimen, which includes maximal debulking surgery, radiation therapy concomitant with temozolomide and maintenance temozolomide (TMZ) concomitant with Optune®, the median overall survival is reported to be 20.9 months from the completion of radiation therapy. There is therefore a clear unmet need for additional effective therapies to treat the disease. Despite numerous clinical studies, no therapy has demonstrated improvement in survival of patients during the past several years.

Although immune checkpoint inhibitors improved survival in several other malignancies, they did not yield a clinically significant outcome in multiple clinical trials in glioblastoma. This was accompanied by lack of effector response induction in patients, potentially owing to a scarcity of T cells within the tumor microenvironment. It could therefore be that an induced increase in cytotoxic T cells within the tumor's microenvironment would result in an improvement in the ability of pembrolizumab to augment the immune response against glioblastoma.

Beyond their anti-mitotic effects in glioblastoma and other cancers, TTFields have been reported to preclinically elicit immunogenic cell death, with expression of the hallmark calreticulin and release of the alarmin high-mobility group box 1 (HMGB-1) and adenosine triphosphate (ATP). This, in turn, led to the recruitment of antigen-presenting cells, such as macrophages and dendritic cells, to their activation and finally to the recruitment of cytotoxic T cells into the tumor. Therefore, it could be theorized that in addition to their direct anti-tumor activity, TTFields would augment the immune response by sensitizing the tumor to pembrolizumab: in the presence of cytotoxic T cells accumulated following TTFields application, pembrolizumab would potentially eliminate tumor's resistance to their activity and therefore lead to effective immune response against the tumor. Indeed, this potential synergy between TTFields and pembrolizumab has already been reported clinically through a single arm, phase 2 study. Maintenance temozolomide, Optune and pembrolizumab were used for the treatment of newly diagnosed glioblastoma patients. The analysis showed that progression-free survival (PFS), overall survival (OS) and two-year OS were significantly longer compared to historical controls who received the regimen without pembrolizumab. Molecular analysis prior to the addition of pembrolizumab confirmed robust T cell activation by TTFields. Subsequently, the ability of the top expanded TCRab clones to adapt to the ever-changing tumor microenvironment through successful clonal switching after the addition of pembrolizumab strongly predicted response to the triple combination.

Moreover, TTFields have been demonstrated to increase the therapeutic effects of radiotherapy (RT) in preclinical models, potentially through effects on the homologous recombination pathway, and inducing a state of ‘BRCAness’ leading to a conditional susceptibility resulting in enhanced sensitivity to ionizing radiation. In two pilot studies TTFields was combined with RT/TMZ, results of these studies demonstrated that TTFields have a high safety profile when combined with radiation therapy in newly diagnosed GBM.

Induction of DNA damage due to radiation treatment exposure, together with downregulation of repair mechanisms by TTFields, induce endoplasmic reticulum (ER) stress. This ER stress, in turn, triggers immunogenic cell death and facilitates the recruitment of immune cells. These intricate processes have the potential to serve as crucial stimuli for the reversal of immunosuppression in patients with GBM. Consequently, this may enhance the efficacy of Pembrolizumab, indicating a synergistic effect between these therapeutic modalities.

### BRIEF SUMMARY

The disclosed study assesses the safety of treatment with TTFields and pembrolizumab during the concomitant chemoradiation treatment in newly diagnosed GBM patients, and to explore the potential for enhancing treatment efficacy.

Disclosed are methods of treating a subject having glioblastoma comprising applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject.

Disclosed are methods of treating a subject having glioblastoma comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject; and wherein the maintenance phase comprises applying an alternating electric field to a target site of the subject for a period of time, wherein the target site comprises one or more glioblastoma cells; administering a therapeutically effective amount of TMZ; and administering a therapeutically effective amount of a checkpoint inhibitor to the subject, wherein the maintenance phase occurs after the treatment phase and after stopping radiation therapy.

Disclosed is a treatment phase dosing regimen comprising applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein each of these steps are applied or administered to the subject within the same 6 weeks.

Disclosed are maintenance phase dosing regimens comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject, wherein no radiation therapy is applied.

Disclosed are methods of increasing survival of a subject having glioblastoma comprising applying an alternating electric field to a target site of the subject for a period of time, wherein the target site comprises one or more glioblastoma cells; administering a radiation therapy; administering a therapeutically effective amount of temozolomide; and

administering a therapeutically effective amount of a checkpoint inhibitor to the subject.

Disclosed are methods of enhancing treatment efficacy of an alternating electric field in a subject having glioblastoma comprising applying an alternating electric field to a target site of the subject for a period of time, wherein the target site comprises one or more glioblastoma cells; administering a radiation therapy; administering a therapeutically effective amount of temozolomide; and administering a therapeutically effective amount of a checkpoint inhibitor to the subject.

Additional advantages of the disclosed methods and compositions will be set forth in part in the description which follows, and in part will be understood from the description, or may be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawing, which is incorporated in and constitutes a part of this specification, illustrates several embodiments of the disclosed methods and compositions and together with the description, serves to explain the principles of the disclosed methods and compositions.

FIG. 1 shows a schematic diagram of a study for testing TTFields with Radiation therapy, Temozolomide, and Pembrolizumab in Newly Diagnosed Glioblastoma patients.

### DETAILED DESCRIPTION

The disclosed methods and compositions may be understood more readily by reference to the following detailed description of particular embodiments and the Example included therein and to the Figure and its previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. Thus, for example, reference to “a checkpoint inhibitor” includes a plurality of such inhibitors, reference to “the checkpoint inhibitor” is a reference to one or more inhibitors and equivalents thereof known to those skilled in the art, and so forth.

The word “or” as used herein means any one member of a particular list and also includes any combination of members of that list.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g., a glioblastoma cancer cell). In some aspects, a “target site” can be a tumor target site.

A “tumor target site” is a site or location within or present on a subject or patient that comprises or is adjacent to one or more glioblastoma cells, previously comprised one or more tumor cells, or is suspected of comprising one or more tumor cells. For example, a tumor target site can refer to a site or location within or present on a subject or patient that is prone to metastases (e.g. brain). Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electric fields delivered to a subject, a sample obtained from a subject or to a specific location within a subject or patient (e.g. a target site). In some aspects, the alternating electrical field can be in a single direction or multiple directions. In some aspects, alternating electric fields can be delivered through two pairs of transducer arrays that generate alternating perpendicular fields within the treated target region. For example, for the Optune™ system (an alternating electric fields delivery system) one pair of electrodes is located to the left and right (LR) of the target region, and the other pair of electrodes is located anterior and posterior (AP) to the target region. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields.

In-vivo and in-vitro studies show that the efficacy of alternating electric fields therapy increases as the intensity of the electric field increases. Therefore, optimizing array placement on the area of a patient's tumor to increase the intensity in the desired region of the tumor can be performed with the Optune system. Array placement optimization may be performed by “rule of thumb” (e.g., placing the arrays on the tumor as close to the desired region of the target site (e.g. cancer cells) as possible), measurements describing the geometry of the patient's tumor, tumor dimensions. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electric field distributes within the head as a function of the positions of the array and, in some aspects, take account for variations in the electrical property distributions within the heads of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field and therapeutically effective amount of a checkpoint inhibitor.

By “treat” is meant to administer or apply a therapeutic, such as radiation therapy, alternating electric fields, temozolomide and/or a checkpoint inhibitor, to a subject, such as a human or other mammal (for example, an animal model), that has cancer or has an increased susceptibility for developing cancer, in order to prevent or delay a worsening of the effects of the cancer, or to partially or fully reverse the effects of the cancer (e.g. glioblastoma).

By “prevent” is meant to minimize the chance that a subject who has an increased susceptibility for developing cancer will develop cancer.

As used herein, the terms “administering” and “administration” refer to any method of providing a therapeutic, such as a checkpoint inhibitor or temozolomide to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, exposing a cancer cell to alternating electrical fields means administering alternating electrical fields to the cancer cell. Thus, in some aspects, exposing a cancer cell to radiation means administering radiation therapy to the cancer cell.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. As used herein, the term “therapeutically effective amount of a checkpoint inhibitor” means an amount of a therapeutic, prophylactic, and/or diagnostic checkpoint inhibitor that is sufficient, when administered in combination with and alternating electric field to a subject suffering from or susceptible to a disease (e.g. glioblastoma), disorder, and/or condition, to treat, alleviate, ameliorate, relieve, alleviate symptoms of, prevent, delay onset of, inhibit progression of, reduce severity of, and/or reduce incidence of the disease, disorder, and/or condition. As used herein, the term “therapeutically effective amount of temozolomide” means an amount of a therapeutic, prophylactic, and/or diagnostic temozolomide that is sufficient, when administered in combination with and alternating electric field to a subject suffering from or susceptible to a disease (e.g. glioblastoma), disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present methods and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

Disclosed are various methods relating to a subject having glioblastoma. In some aspects, the subject has been newly diagnosed with glioblastoma (according to WHO 2021 Classification). Thus, in some aspects, the subject has never undergone a previous glioblastoma treatment. In some aspects, the subject is a candidate for standard chemotherapy and radiation therapy and/or alternating electrical field therapy. Thus, in some aspects, the subject has been determined (by a physician) to be a candidate for a standard glioblastoma therapy of chemoradiation followed by alternating electric field concomitant with Temozolomide. In some aspects, the subject has undergone surgery. For example, in some aspects, the surgery is a tumor resection or debulking surgery. In some aspects, the subject has undergone a biopsy, such as a brain biopsy on a glioblastoma. In some aspects, the subject has been treated with a corticosteroid for at least 5 days prior to radiation therapy. In some aspects, if the subject has been treated with a corticosteroid, they have not exceeded the maximum dose of corticosteroid treatment. For example, a subject can be treated with a maximum dose of 4 mg/day dexamethasone or equivalent doses for other corticosteroids, which has been stable or decreased for ≥5 days prior to start of radiotherapy. In some aspects, the subject has a life expectancy of at least 3 months. Thus, in some aspects, the disclosed methods are not used for subjects with a life expectancy of less than 3 months. In some aspects, the subject does not have an acute viral, bacterial, or fungal infection. In some aspects, the disclosed methods are a first line treatment for a subject having glioblastoma. In some aspects, the subject has not received prior chemotherapy for a brain tumor or prior radiation therapy to the brain. In some aspects, the subject has not previously treated with TMZ. Thus, in some aspects, the subject has not previously received any of the treatments of the disclosed method alone or in combination.

In some aspects, the subject's survival is longer compared to an age-matched subject treated with the standard therapy of concomitant radio-chemotherapy, followed by alternating electric field and maintenance TMZ, without a checkpoint inhibitor (e.g. Pembrolizumab). In some aspects, the subject's survival is longer compared to an age-matched subject treated with TMZ, a checkpoint inhibitor (e.g. Pembrolizumab), and radiation. In some aspects, the subject's survival is longer compared to an age-matched subject treated with standard therapy of chemotherapy and radiation therapy, without alternating electric field.

### 1. Methods of Treating Glioblastoma

Disclosed are methods of treating a subject having glioblastoma comprising applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject.

In some aspects, the disclosed methods of treating are the first line of glioblastoma treatment for a subject having glioblastoma. Thus, in some aspects, the subject having glioblastoma being administered the alternating electric field, radiation therapy, TMZ, and checkpoint inhibitor have not yet received any other glioblastoma treatment.

In some aspects, disclosed are methods of treating a subject having glioblastoma comprising a treatment phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject. In some aspects, a treatment phase is a period of time in which each of the alternating electric field, radiation therapy, TMZ and checkpoint inhibitor are administered to a subject. In some aspects, the treatment phase can be six weeks. In some aspects, the treatment phase can be four to eight weeks. Thus, in some aspects, a single dosing regimen for each of the alternating electric field, radiation therapy, TMZ and checkpoint inhibitor is applied within the treatment phase. In some aspects, any of the dosing regimens for a treatment phase disclosed herein can be applied.

In some aspects, disclosed are methods of treating a subject having glioblastoma comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject; and wherein the maintenance phase comprises applying an alternating electric field to a target site of the subject for a period of time, wherein the target site comprises one or more glioblastoma cells; administering a therapeutically effective amount of TMZ; and administering a therapeutically effective amount of a checkpoint inhibitor to the subject, wherein the maintenance phase occurs after the treatment phase and after stopping the administration of the radiation therapy. In some aspects, a maintenance phase is a period of time in which each of the alternating electric field, TMZ and checkpoint inhibitor are administered to a subject without radiation therapy being administered. In some aspects, administration of TMZ is delayed during the maintenance phase. For example, after ending radiation therapy and thus beginning the maintenance phase, TMZ administration may be paused for about 1, 2, 3, 4, 5, or 6 weeks. In some aspects, 1, 2, 3, 4, 5, or 6 weeks into the maintenance phase, TMZ administration can begin. Thus, in some aspects, a maintenance phase is a period of time in which each of the alternating electric field and checkpoint inhibitor are administered to a subject without radiation therapy being administered.

In some aspects, the maintenance phase occurs after stopping radiation therapy (e.g. the maintenance phase does not include administering a radiation therapy). In some aspects, the treatment phase is six weeks. In some aspects, the maintenance phase occurs after the administration of the combination of alternating electric field, radiation therapy, TMZ and checkpoint inhibitor. In some aspects, the maintenance phase can be 1, 2, 3, 4, 5, 6, 7, 8, 9, 10, 11, or 12 months. In some aspects, the maintenance phase can be 1, 2, 3, 4, or 5 years. In some aspects, the maintenance phase is a maximum of 2 years (e.g., 35 cycles of checkpoint inhibitor). In some aspects, applying the alternating electric field, administration of a therapeutically effective amount of TMZ or administration of a therapeutically effective amount of a checkpoint inhibitor to the subject in the maintenance phase can occur for different lengths of time. For example, in some aspects, applying the alternating electric field to a target site of the subject during the maintenance phase can continue until the earlier of second disease progression or 24 months from enrollment in the treatment. In some aspects, administration of a therapeutically effective amount of a checkpoint inhibitor to the subject during the maintenance phase can include up to 35 treatments including those administered during the treatment phase. Thus, in some aspects, a single dosing regimen within the maintenance phase comprises each of the alternating electric field, TMZ and checkpoint inhibitor, without radiation therapy. Thus, in some aspects, a single dosing regimen within the maintenance phase consists of each of the alternating electric field, TMZ and checkpoint inhibitor, without radiation therapy. In some aspects, any of the dosing regimens for a maintenance phase disclosed herein can be applied.

In some aspects, disclosed are methods of treating a subject having glioblastoma comprising a treatment phase and a maintenance phase, the method comprising, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein the radiation therapy is administered (60 Gy in 30 fractions) for 6 weeks; and the a checkpoint inhibitor is administered every 3 weeks (21 days); and the alternating electric field is applied continuously without interruption until second disease progression or 24 months from enrollment (the earlier of the two); and TMZ is administered at a dose of 75 mg/m 2 /day for 6 weeks. In some aspects, after the radiation therapy is administered for 6 weeks, radiation therapy is stopped and is not resumed. In some aspects, the checkpoint inhibitor is continually administered every 3 weeks without interruption for up to 35 treatments (e.g., the checkpoint inhibitor administration does not stop when radiation therapy is stopped). In some aspects, the checkpoint inhibitor is continually administered every 3 weeks without interruption for up to 35 treatments or until disease progresses. In some aspects, TMZ is administered for 6 weeks, then the subject is given a 4 to 6 week break. In some aspects, 4 to 6 weeks after the first 6 weeks of TMZ (e.g. 4 to 6 weeks after the radiation therapy), TMZ can be administered at a dose of 150-200 mg/m 2 for 5 days during each 28-day cycle for up to 6 chemotherapy cycles. In some aspects, everything that is administered during the 6 week radiation therapy is considered the treatment phase. In some aspects, any additional application of the alternating electric field, administration of checkpoint inhibitors, and/or administration of and TMZ once radiation therapy is stopped can be considered a maintenance phase. In some aspects, the maintenance phase is comprises the application of the alternating electric field and administration of checkpoint inhibitors but no TMZ. For example, in some aspects, a subject can be given a break from TMZ administration for about 1 to 6 weeks after treatment phase, meaning that the first 1 to 6 weeks of the maintenance phase can be the application of the alternating electric field and administration of checkpoint inhibitors but no TMZ. In some aspects, the maintenance phase is comprises the application of the alternating electric field, administration of checkpoint inhibitors, and administration of TMZ.

In some aspects, “enrollment,” as used throughout, refers to subjects being enrolled up to 6 weeks of signing the informed consent form, following the completion of screening/baseline procedures, and only after verifying that the subject meets the eligibility criteria. Date of enrollment is defined as the date the subject met all eligibility criteria and was planned to start study treatment. In some aspects, subjects begin treatment with alternating electric field, checkpoint inhibitor (e.g., Pembrolizumab), radiation therapy, and TMZ within 14 days of enrollment, and no later than 6 weeks from surgery or biopsy. In some aspects, checkpoint inhibitor and alternating electric field can be initiated ±3 days of each other and radiation therapy/TMZ.

### 2. Methods of Increasing Survival of a Subject

Disclosed are methods of increasing survival of a subject having glioblastoma using one or more of the disclosed dosing regimens.

Disclosed are methods of increasing survival of a subject having glioblastoma comprising applying an alternating electric field to a target site of the subject for a period of time, wherein the target site comprises one or more glioblastoma cells; administering a radiation therapy; administering a therapeutically effective amount of temozolomide; and administering a therapeutically effective amount of a checkpoint inhibitor to the subject.

In some aspects, the subject's survival is increased compared to an age-matched subject treated with the standard therapy of concomitant radio-chemotherapy, followed by alternating electric field and maintenance TMZ, without a checkpoint inhibitor (e.g. Pembrolizumab). In some aspects, the subject's survival is longer compared to an age-matched subject treated with TMZ, a checkpoint inhibitor (e.g. Pembrolizumab), and radiation. In some aspects, the subject's survival is longer compared to an age-matched subject treated with standard therapy of chemotherapy and radiation therapy, without alternating electric field.

In some aspects, disclosed are methods increasing survival of a subject having glioblastoma comprising a treatment phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject. In some aspects, a treatment phase is a period of time in which each of the alternating electric field, radiation therapy, TMZ and checkpoint inhibitor are administered to a subject. In some aspects, the treatment phase can be six weeks. In some aspects, the treatment phase can be four to eight weeks. Thus, in some aspects, a single dosing regimen for each of the alternating electric field, radiation therapy, TMZ and checkpoint inhibitor is applied within the treatment phase. In some aspects, any of the dosing regimens for a treatment phase disclosed herein can be applied.

In some aspects, disclosed are methods of increasing survival of a subject having glioblastoma comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject; and wherein the maintenance phase comprises applying an alternating electric field to a target site of the subject for a period of time, wherein the target site comprises one or more glioblastoma cells; administering a therapeutically effective amount of TMZ; and administering a therapeutically effective amount of a checkpoint inhibitor to the subject, wherein the maintenance phase occurs after the treatment phase and after stopping administration of the radiation therapy. In some aspects, a maintenance phase is a period of time in which each of the alternating electric field, TMZ and checkpoint inhibitor are administered to a subject without radiation therapy being administered. In some aspects, administration of TMZ is delayed during the maintenance phase. For example, after ending radiation therapy and thus beginning the maintenance phase, TMZ administration may be paused for about 1, 2, 3, 4, 5, or 6 weeks. In some aspects, 1, 2, 3, 4, 5, or 6 weeks into the maintenance phase, TMZ administration can begin. Thus, in some aspects, a maintenance phase is a period of time in which each of the alternating electric field and checkpoint inhibitor are administered to a subject without radiation therapy being administered. Thus, in some aspects, the maintenance phase occurs after stopping radiation therapy (e.g. the maintenance phase does not include administering a radiation therapy). In some aspects, the treatment phase is six weeks. In some aspects, the maintenance phase occurs after the administration of the combination of alternating electric field, radiation therapy, TMZ and checkpoint inhibitor. In some aspects, the maintenance phase can be 1, 2, 3, 4, 5, 6, 7, 8, 9, 10, 11, or 12 months. In some aspects, the maintenance phase can be 1, 2, 3, 4, or 5 years. In some aspects, the maintenance phase is a maximum of 2 years (e.g., 35 cycles of checkpoint inhibitor). In some aspects, applying the alternating electric field, administration of a therapeutically effective amount of TMZ or administration of a therapeutically effective amount of a checkpoint inhibitor to the subject in the maintenance phase can occur for different lengths of time. For example, in some aspects, applying the alternating electric field to a target site of the subject during the maintenance phase can continue until the earlier of second disease progression or 24 months from enrollment in the treatment. In some aspects, administration of a therapeutically effective amount of a checkpoint inhibitor to the subject during the maintenance phase can include up to 35 treatments including those administered during the treatment phase. Thus, in some aspects, a single dosing regimen within the maintenance phase comprises each of the alternating electric field, TMZ and checkpoint inhibitor, without radiation therapy. in some aspects, a single dosing regimen within the maintenance phase consists of each of the alternating electric field, TMZ and checkpoint inhibitor, without radiation therapy. In some aspects, any of the dosing regimens for a maintenance phase disclosed herein can be applied.

In some aspects, disclosed are methods of increasing survival of a subject comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein the radiation therapy is administered (60 Gy in 30 fractions) for 6 weeks; and the a checkpoint inhibitor is administered every 3 weeks (21 days); and the alternating electric field is applied continuously without interruption until second disease progression or 24 months from enrollment (the earlier of the two); and TMZ is administered at a dose of 75mg/m2/day for 6 weeks. In some aspects, after the radiation therapy is administered for 6 weeks, radiation therapy is stopped and is not resumed. In some aspects, the checkpoint inhibitor is continually administered every 3 weeks without interruption for up to 35 treatments (e.g., the checkpoint inhibitor administration does not stop when radiation therapy is stopped). In some aspects, TMZ is administered for 6 weeks, then the subject is given a 4 to 6 week break. In some aspects, 4 to 6 weeks after the first 6 weeks of TMZ (e.g. 4 to 6 weeks after the radiation therapy), TMZ can be administered at a dose of 150-200 mg/m2 for 5 days during each 28-day cycle for up to 6 chemotherapy cycles. In some aspects, everything that is administered during the 6 week radiation therapy is considered the treatment phase. In some aspects, any additional application of the alternating electric field, administration of checkpoint inhibitors, and/or administration of and TMZ once radiation therapy is stopped can be considered a maintenance phase. In some aspects, the maintenance phase is comprises the application of the alternating electric field and administration of checkpoint inhibitors but no TMZ. For example, in some aspects, a subject can be given a break from TMZ administration for about 1 to 6 weeks after treatment phase, meaning that the first 1 to 6 weeks of the maintenance phase can be the application of the alternating electric field and administration of checkpoint inhibitors but no TMZ. In some aspects, the maintenance phase is comprises the application of the alternating electric field, administration of checkpoint inhibitors, and administration of TMZ.

In some aspects, “enrollment,” as used throughout, refers to subjects being enrolled up to 6 weeks of signing the informed consent form, following the completion of screening/baseline procedures, and only after verifying that the subject meets the eligibility criteria. Date of enrollment is defined as the date the subject met all eligibility criteria and was planned to start study treatment. In some aspects, subjects begin treatment with alternating electric field, checkpoint inhibitor (e.g., Pembrolizumab), radiation therapy, and TMZ within 14 days of enrollment, and no later than 6 weeks from surgery or biopsy. In some aspects, checkpoint inhibitor and alternating electric field can be initiated ±3 days of each other and radiation therapy/TMZ.

### 3. Methods of Enhancing Treatment Efficacy

Disclosed are methods of enhancing treatment efficacy of an alternating electric field in a subject having glioblastoma comprising applying an alternating electric field to a target site of the subject for a period of time, wherein the target site comprises one or more glioblastoma cells; administering a radiation therapy; administering a therapeutically effective amount of temozolomide; and administering a therapeutically effective amount of a checkpoint inhibitor to the subject.

In some aspects, the disclosed methods of enhancing treatment efficacy of an alternating electric field in a subject having glioblastoma are the first line of glioblastoma treatment for a subject having glioblastoma. Thus, in some aspects, the subject having glioblastoma being administered the alternating electric field, radiation therapy, TMZ, and checkpoint inhibitor have not yet received any other glioblastoma treatment.

In some aspects, disclosed are methods of enhancing treatment efficacy of an alternating electric field in a subject having glioblastoma comprising a treatment phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject. In some aspects, a treatment phase is a period of time in which each of the alternating electric field, radiation therapy, TMZ and checkpoint inhibitor are administered to a subject. In some aspects, the treatment phase can be six weeks. In some aspects, the treatment phase can be four to eight weeks. Thus, in some aspects, a single dosing regimen for each of the alternating electric field, radiation therapy, TMZ and checkpoint inhibitor is applied within the treatment phase. In some aspects, any of the dosing regimens for a treatment phase disclosed herein can be applied.

In some aspects, disclosed are methods enhancing treatment efficacy of an alternating electric field in a subject having glioblastoma comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject; and wherein the maintenance phase comprises applying an alternating electric field to a target site of the subject for a period of time, wherein the target site comprises one or more glioblastoma cells; administering a therapeutically effective amount of TMZ; and administering a therapeutically effective amount of a checkpoint inhibitor to the subject, wherein the maintenance phase occurs after the treatment phase and after stopping radiation therapy. In some aspects, a maintenance phase is a period of time in which each of the alternating electric field, TMZ and checkpoint inhibitor are administered to a subject without radiation therapy being administered. In some aspects, administration of TMZ is delayed during the maintenance phase. For example, after ending radiation therapy and thus beginning the maintenance phase, TMZ administration may be paused for about 1, 2, 3, 4, 5, or 6 weeks. In some aspects, 1, 2, 3, 4, 5, or 6 weeks into the maintenance phase, TMZ administration can begin. Thus, in some aspects, a maintenance phase is a period of time in which each of the alternating electric field and checkpoint inhibitor are administered to a subject without radiation therapy being administered.

In some aspects, the maintenance phase occurs after stopping the administration of the radiation therapy (e.g. the maintenance phase does not include administering a radiation therapy). In some aspects, the treatment phase is four to six weeks. In some aspects, the maintenance phase occurs after the administration of the combination of alternating electric field, radiation therapy, TMZ and checkpoint inhibitor. In some aspects, the maintenance phase can be 1, 2, 3, 4, 5, 6, 7, 8, 9, 10, 11, or 12 months. In some aspects, the maintenance phase can be 1, 2, 3, 4, or 5 years. In some aspects, the maintenance phase is a maximum of 2 years (e.g., 35 cycles of checkpoint inhibitor). In some aspects, applying the alternating electric field, administration of a therapeutically effective amount of TMZ or administration of a therapeutically effective amount of a checkpoint inhibitor to the subject in the maintenance phase can occur for different lengths of time. For example, in some aspects, applying the alternating electric field to a target site of the subject during the maintenance phase can continue until the earlier of second disease progression or 24 months from enrollment in the treatment. In some aspects, administration of a therapeutically effective amount of a checkpoint inhibitor to the subject during the maintenance phase can include up to 35 treatments including those administered during the treatment phase. Thus, in some aspects, a single dosing regimen within the maintenance phase comprises each of the alternating electric field, TMZ and checkpoint inhibitor, without radiation therapy. In some aspects, a single dosing regimen within the maintenance phase consists of each of the alternating electric field, TMZ and checkpoint inhibitor, without radiation therapy. In some aspects, any of the dosing regimens for a maintenance phase disclosed herein can be applied.

In some aspects, disclosed are methods of enhancing treatment efficacy comprising a treatment phase and a maintenance phase, wherein the treatment phase comprises applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein the radiation therapy is administered (60 Gy in 30 fractions) for 6 weeks; and the a checkpoint inhibitor is administered every 3 weeks (21 days); and the alternating electric field is applied continuously without interruption until second disease progression or 24 months from enrollment (the earlier of the two); and TMZ is administered at a dose of 75mg/m2/day for 6 weeks. In some aspects, after the radiation therapy is administered for 6 weeks, radiation therapy is stopped and is not resumed. In some aspects, the checkpoint inhibitor is continually administered every 3 weeks without interruption for up to 35 treatments (e.g., the checkpoint inhibitor administration does not stop when radiation therapy is stopped). In some aspects, TMZ is administered for 6 weeks, then the subject is given a 4 to 6 week break. In some aspects, 4 to 6 weeks after the first 6 weeks of TMZ (e.g. 4 to 6 weeks after the radiation therapy), TMZ can be administered at a dose of 150-200 mg/m2 for 5 days during each 28-day cycle for up to 6 chemotherapy cycles. In some aspects, everything that is administered during the 6 week radiation therapy is considered the treatment phase. In some aspects, any additional application of the alternating electric field or administration of checkpoint inhibitors or TMZ once radiation therapy is stopped can be considered a maintenance phase. In some aspects, any additional application of the alternating electric field or administration of checkpoint inhibitors and TMZ once radiation therapy is stopped can be considered a maintenance phase. In some aspects, the maintenance phase is comprises the application of the alternating electric field and administration of checkpoint inhibitors but no TMZ. For example, in some aspects, a subject can be given a break from TMZ administration for about 1 to 6 weeks after treatment phase, meaning that the first 1 to 6 weeks of the maintenance phase can be the application of the alternating electric field and administration of checkpoint inhibitors but no TMZ. In some aspects, the maintenance phase is comprises the application of the alternating electric field, administration of checkpoint inhibitors, and administration of TMZ.

In some aspects, “enrollment,” as used throughout, refers to subjects being enrolled up to 6 weeks of signing the informed consent form, following the completion of screening/baseline procedures, and only after verifying that the subject meets the eligibility criteria. Date of enrollment is defined as the date the subject met all eligibility criteria and was planned to start study treatment. In some aspects, subjects begin treatment with alternating electric field, checkpoint inhibitor (e.g., Pembrolizumab), radiation therapy, and TMZ within 14 days of enrollment, and no later than 6 weeks from surgery or biopsy. In some aspects, checkpoint inhibitor and alternating electric field can be initiated ±3 days of each other and radiation therapy/TMZ.

### C. Alternating Electric Fields

The methods disclosed herein comprise applying an alternating electric field. In some aspects, the alternating electric field used in the methods disclosed herein is a tumor-treating field (TTFields). Alternating electric fields are a non-invasive, regional antimitotic treatment modality with minimal toxicity which has been approved for the treatment of recurrent and newly diagnosed glioblastoma (GBM) and for unresectable malignant pleural mesothelioma by the Food and Drug Administration (FDA) and has obtained a CE mark in Europe for the same indication.

Alternating electric fields act by delivering low intensity (1-3 V/cm), intermediate frequency (100-300 kHz), alternating electric fields to the tumor using non-invasive transducer arrays placed on the skin around the region of the body containing the tumor. Alternating electric fields act predominantly during two phases of mitosis: 1) during metaphase, by disrupting the formation of the mitotic spindle, and 2) during cytokinesis, by dielectrophoretic dislocation of intracellular constituents resulting in apoptosis. In addition, it has been shown in GBM and non-small cell lung cancer (NSCLC) models that alternating electric fields also induce formation of DNA double-strand breaks (DSB) and impair DNA damage repair (DDR) mechanisms. The efficacy of alternating electric fields is frequency-dependent on specific cell types. The anti-mitotic effect of alternating electric fields has been shown in multiple cell lines when the appropriate frequency was utilized. This includes but is not limited to the following tumor models: GBM at 200 kHz, NSCLC at 150 kHz; breast carcinoma at 150 kHz; melanoma at 100 kHz.

The effect of alternating electric fields is directional, i.e., alternating electric fields are most effective when applied in the direction of the division axis of the dividing cell. To increase the efficacy of alternating electric fields, two sequential field directions can be applied to tumors by using two perpendicular pairs of transducer arrays. Using two-directional alternating electric fields in pilot clinical testing demonstrated alternating electric fields to be biologically active in human tumors.

In some aspects, the alternating electric field can vary dependent on the type of cell or condition to which the alternating electric field is applied. In some aspects, the alternating electric field can be applied through one or more electrodes placed on or in the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be about 50 kHz, 100 kHz, 200 kHz, 300 kHz, 400 kHz, 500 kHz, or any frequency between. In some aspects, the frequency of the alternating electric field is from about 200 kHz to about 400 kHz, from about 250 kHz to about 350 kHz, and may be about 150 kHz, about 200 kHz, or about 300 kHz.

In some aspects, the field strength of the alternating electric fields can be between 1 and 4 V/cm RMS. In some aspects, different field strengths can be used (e.g., between 0.1 and 10 V/cm). In some aspects, the field strength can be about 1.75 V/cm RMS. In some embodiments the field strength is at least 1 V/cm. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two-hour duration.

In some aspects, the exposure may last for at least 6 hours, at least 12 hours, at least 24 hours, at least 36 hours, at least 48 hours, or at least 72 hours or more. In some aspects, the exposure can be consecutive or cumulative. In some aspects, the consecutive exposure may last for at least 6 hours, at least 12 hours, at least 24 hours, at least 36 hours, at least 48 hours, or at least 72 hours or more. In some aspects, the cumulative exposure may last for at least 42 hours, at least 84 hours, at least 168 hours, at least 250 hours, at least 400 hours, at least 500 hours, at least 750 hours, or more. In some aspects, there can be a break in treatment and the alternating electric fields are applied at least 50%, 60%, 70%, or 80% of treatment time. For example, in some aspects, cumulative exposure can be for at least 12 hours in a period of 24 hours or at least 18 hours in a period of 24 hours.

In some aspects, the alternating electric field is applied continuously. Continuous application refers to applying the alternating field for at least 12 hours in a 24 hour period, for example at least 15 hours in a 12 hour period or at least 18 hours in a 24 hour period. Thus, continuous application includes, for example, applying the alternating electric field for 18 hours, then stopping for 6 hours, then continuing application of the alternating electric field. In some aspects there is no interruption from the alternating electric field between a treatment phase and a maintenance phase. In some aspects, an interruption can be a period of time greater than 1, 2, 3, 4, 5, 6, or 7 days without application of the alternating electric field and is not considered part of the treatment duration. Thus, in some aspects, a 6 hour cessation of the alternating electric field is part of the treatment and not considered an interruption. In some aspects, an interruption can include applying no alternating electric field for a week, 5 days, 3 days, 2 days or 1 day. In some aspects, the alternating electric field is applied continuously until second disease progression or 24 months from enrollment. In some aspects, the alternating electric field has a frequency and field strength. In some aspects, the alternating electric field has a frequency between 50 kHz and 1 MHz. In some aspects, the alternating electric field has a frequency of about 150 to 250 kHz. In some aspects, the alternating electric field has a frequency of 200 kHz. In some aspects, the alternating electric field has any of the frequencies described throughout. In some aspects, the alternating electric field has a field strength of between 0.5 and 10 V/cm RMS. In some aspects, the alternating electric field has a field strength of about 0.9 V/cm RMS. In some aspects, the alternating electric field has any of the field strengths described throughout.

In some aspects, the alternating electric field is applied continuously throughout the treatment phase and the maintenance phase.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

In some aspects, the exposure may last for at least 6 hours, at least 12 hours, at least 24 hours, at least 36 hours, at least 48 hours, or at least 72 hours or more.

In addition, when the alternating electric field is applied to a subject, the period of time that the alternating electric field is applied may be a continuous period of time or a cumulative period of time. That is, the period of time that the alternating electric field is applied may include a single session (i.e., continuous application) as well as multiple sessions with minor breaks in between sessions (i.e., consecutive applications for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.). For example, the alternating electric field can be applied for at least 12 hours, 16 hours, or 18 hours cumulative each day for a week, a month, two months, three months, etc.

### 1. TTFields in Glioblastoma

Based on clinical data in recurrent GBM and a pilot study in newly diagnosed GBM with Optune™ in combination with temozolomide (TMZ) that demonstrated favorable safety profile and promising efficacy, an international phase III study in newly diagnosed GBM, evaluating the role of TTFields in combination with temozolomide maintenance after surgery and chemoradiation versus temozolomide alone was conducted. Patients were randomized (at a 1:2 ratio) to either standard adjuvant TMZ alone (150-200 mg/m²/d×5d) or TMZ and continuous administration of TTFields. A total of 695 patients were randomized. The median progression-free survival was 6.7 months for the patients treated with TTFields/TMZ versus 4.0 months for TMZ alone (HR 0.63; 95% CI 0.52-0.76; p<0.001). Median overall survival from randomization was 20.9 months versus 16 months for TTFields/TMZ and TMZ alone, respectively, with a hazard ratio of 0.63(95 % CI 0.53-0.76), p<0.001.

The most common adverse events in the TTFields/TMZ arm, defined as occurring in ≥10% of patients, were thrombocytopenia, nausea, constipation, vomiting, fatigue, medical device site reaction, headache, convulsions, and depression. Grade 3 to 4 adverse events were well balanced between the 2 treatment arms. None of the systemic grade 3 to 4 adverse events were considered related to TTFields by any of the investigators. Mild to moderate skin toxicity underneath the transducer arrays occurred in 52% of patients who received TTFields-temozolomide vs no patients who received temozolomide alone.

Based on the data submitted to FDA from this newly diagnosed GBM study, FDA approved Optune™ together with concomitant temozolomide for the treatment of adult patients with newly diagnosed GBM on Oct. 5, 2015 (see Optune's IFU on https://www.optune.com/Content/pdfs/Optune_IFU_8.5x11.pdf). In the US, Optune™ with TMZ is indicated for the treatment of adult patients with newly diagnosed, supratentorial glioblastoma following maximal debulking surgery and completion of radiation therapy together with concomitant standard of care chemotherapy.

### D. Checkpoint Inhibitors

The methods disclosed herein comprise administering a checkpoint inhibitor. In some aspects, the checkpoint inhibitor used in a method disclosed herein can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In some aspects, the checkpoint inhibitor used in a method disclosed herein is pembrolizumab. Pembrolizumab is a potent humanized immunoglobulin G4 (IgG4) monoclonal antibody (mAb) with high specificity of binding to the programmed cell death 1 (PD-1) receptor, thus inhibiting its interaction with programmed cell death ligand 1 (PD-L1) and programmed cell death ligand 2 (PD-L2). In some aspects, pembrolizumab has high affinity and potent receptor blocking activity for PD-1.

In some aspects, the checkpoint inhibitor is administered once every 1, 2, 3, 4, 5, or 6 weeks. In some aspects, the checkpoint inhibitor is administered once every 3 weeks. For example, in some aspects, the pembrolizumab is administered once every 3 weeks. In some aspects, the checkpoint inhibitor is administered at 100-800 mg per dose. In some aspects, the checkpoint inhibitor is administered at 200 mg per dose. For example, in some aspects, the pembrolizumab is administered at 200 mg per dose. In some aspects, the checkpoint inhibitor is administered intravenously (IV) or orally. For example, in some aspects, the pembrolizumab is administered IV.

In some aspects, a treatment phase dosing regimen can comprise the checkpoint inhibitor administered as disclosed herein. For example, in some aspects, a treatment phase dosing regimen can comprise administering pembrolizumab at 200 mg per dose. In some aspects, the treatment phase can be 6 weeks and the checkpoint inhibitor can be administered twice during the 6 week treatment phase.

In some aspects, the dosing regimen for the checkpoint inhibitor is the same during the treatment phase as it is during the maintenance phase. In some aspects, the maintenance phase of the treatment can begin at least 1, 2, 3, 4, 5, 6, 7, or 8 weeks after the end of the concomitant radiation therapy (e.g. treatment phase). In some aspects, the maintenance phase of the dosing regimen can begin at least 1 day after the treatment phase and once the radiation therapy is stopped. For example, the checkpoint inhibitor can continue every 3 weeks without interruption between the treatment to maintenance phases. In some aspects, the checkpoint inhibitor can be administered for up to 35 treatments. Thus, in some aspects, there is no interruption in administering the checkpoint inhibitor between the treatment phase and maintenance phase.

### E. Temozolomide (TMZ)

The methods disclosed herein comprise administering temozolomide (TMZ). In some aspects, the TMZ used in the methods disclosed herein is administered daily at 75 mg/m² per dose for 6 weeks (e.g., the 6 weeks concomitant with radiation therapy) and/or at 150-200 mg/m² for five days every 28 days.

In some aspects, the TMZ is administered at 50-100 mg/m² per dose. In some aspects, the TMZ is administered at 75 mg/m² per dose.

In some aspects, a treatment phase dosing regimen can comprise TMZ administered as disclosed herein. For example, in some aspects, a treatment phase dosing regimen can comprise administering TMZ at 75 mg/m2 per dose. In some aspects, a treatment phase dosing regimen can comprise administering TMZ at 50-100 mg/m² per dose. In some aspects, the treatment phase dosing regimen comprises a daily dose of TMZ for 6 weeks.

In some aspects, the TMZ is administered concomitant to the radiation therapy. In some aspects, this concomitant administration of TMZ and radiation therapy occurs during the treatment phase along with applying alternating electric fields and administering a checkpoint inhibitor. In some aspects, during the concomitant administration (or treatment phase) subjects can receive TMZ at a dose of 75mg/m² daily for 4 to 8 weeks, more specifically for 6 weeks. For example, in some aspects, a treatment phase dosing regimen can comprise administering TMZ at a dose of 75mg/m² daily for 6 weeks.

In some aspects, the method comprises a maintenance TMZ treatment following the concomitant radiation therapy and TMZ. In some aspects, the maintenance TMZ treatment occurs after radiation therapy has stopped. In some aspects, the maintenance TMZ treatment can begin at least 1, 2, 3, 4, 5, 6, 7, or 8 weeks after the end of the concomitant radiation therapy and TMZ. In some aspects, the maintenance TMZ treatment can begin 4 to 6 weeks after the end of the concomitant radiation therapy and TMZ. For example, in some aspects, the maintenance TMZ treatment can begin at least 4 weeks after the end of the concomitant radiation therapy and TMZ. In some aspects, maintenance TMZ treatment can be administered for 5 days every 28 days at a dose of 150-200 mg/m² daily. In some aspects, the maintenance TMZ treatment occurs for 5 days during each 28-day cycle for up to 6 chemotherapy cycles. In some aspects, a chemotherapy cycle is 28 consecutive days, the count starts from the first day of administering maintenance TMZ (4 weeks from end of radiotherapy).

Thus, in some aspects, maintenance TMZ treatment can be administered 5 days every 28 days at a dose of 150-200 mg/m² daily for up to 6 chemotherapy cycles. In some aspects, TMZ, both concomitant with radiation therapy (e.g. treatment phase) or during the maintenance treatment, is administered orally. In some aspects, a maintenance phase dosing regimen can comprise TMZ administered after stopping radiation therapy as disclosed herein.

### F. Radiation Therapy

The methods disclosed herein comprise administering radiation therapy, for example during a treatment phase. In some aspects, the radiation therapy in the methods disclosed herein is administered concomitant to the TMZ administration for a period of time. In some aspects, the radiation therapy is administered daily for six weeks. In some aspects, the radiation therapy is administered 5 days a week for six weeks. In some aspects, the total dose of radiation therapy is about 60 Gy. Thus, in some aspects, radiation therapy is administered at 2 Gy/day for 5 days/week for 6 weeks.

In some aspects, radiation therapy can be administered to the subject for 4 to 8 weeks.

In some aspects, there is no radiation therapy administered during the maintenance phase.

### G. Dosing Regimens

Disclosed are dosing regimens suitable to be used in any of the disclosed methods. Disclosed are dosing regimens for a treatment phase and dosing regimens for a maintenance phase. Also disclosed are dosing regimens that combine the treatment phase dosing regimen and the maintenance phase dosing regimen.

In some aspects, a single dosing regimen for each of the alternating electric field, radiation therapy, TMZ and checkpoint inhibitor is applied within the treatment phase. Therefore, in some aspects, a single treatment phase dosing regimen can be four weeks or six weeks.

In some aspects, a single dosing regimen for each of the alternating electric field, TMZ and checkpoint inhibitor, without radiation, is applied within the maintenance phase. Therefore, in some aspects, a single maintenance phase dosing regimen can be four weeks. In some aspects, a single maintenance phase dosing regimen can be administered every month for 1, 2, 3, 4, 5, 6, or more months.

Disclosed are dosing regimens comprising applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein each of these steps are applied or administered to the subject for 6 weeks. In some aspects, the dosing regimen can be 4 to 8 weeks. In some aspects, this dosing regimen that comprises radiation therapy can be referred to as the treatment phase dosing regimen. Thus, disclosed is a treatment phase dosing regimen comprising applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein each of these steps are applied or administered to the subject for 4 to 6 weeks.

Disclosed are dosing regimens comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject, wherein no radiation therapy is applied. In some aspects, this dosing regimen can be referred to as the maintenance phase dosing regimen as the maintenance phase does not include administering radiation therapy. In fact, in some aspects, the maintenance phase occurs after stopping radiation therapy (e.g. from the treatment phase). In some aspects, a single maintenance phase dosing regimen takes place over a four week period.

Thus, disclosed are maintenance phase dosing regimens comprising applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject, wherein no radiation therapy is applied.

In some aspects, the TMZ is administered daily concomitant with the radiation therapy. In some aspects, the TMZ is administered at 50-100 mg/m² per dose. In some aspects, the TMZ is administered at 75 mg/m² per dose (e.g., 75 mg/m²/day). Thus, in some aspects, a treatment phase dosing regimen can comprise TMZ administered at 50-100 mg/m² per dose, specifically at 75 mg/m² per dose. For example, in some aspects, a treatment phase dosing regimen can comprise administering TMZ at 75 mg/m²/day for 6 weeks.

In some aspects, the TMZ is administered concomitant to the radiation therapy. In some aspects, this concomitant administration of TMZ and radiation therapy occurs during the treatment phase along with applying alternating electric fields and administering a checkpoint inhibitor. In some aspects, during the concomitant administration (or treatment phase) subjects can receive TMZ at a dose of 75mg/m2 daily for 6 weeks.

In some aspects, the dosing regimen can be a maintenance dosing regimen. In some aspects, the maintenance phase dosing regimen occurs after radiation therapy from the treatment dosing regimen has stopped. In some aspects, the maintenance phase dosing regimen can begin at least 1 day after the end of the concomitant radiation therapy (e.g. treatment dosing regimen). In some aspects, the maintenance phase dosing regimen can begin at least 1, 2, 3, 4, 5, 6, 7, or 8 weeks after the end of the concomitant radiation therapy (e.g. treatment dosing regimen). For example, in some aspects, the administration of TMZ during the maintenance phase dosing regimen can begin at least 4 weeks after the end of the concomitant radiation therapy and TMZ (e.g. treatment dosing regimen). Thus, in some aspects, a subject can have a 1 to 6 week, preferably 4 week, break from TMZ administration that occurs the first 1 to 6 weeks of the maintenance phase. In some aspects, TMZ in the maintenance phase dosing regimen can be administered 5 days every 28 days at a dose of 150-200 mg/m² daily. In some aspects, the maintenance phase dosing regimen occurs for 4, 6, 8, 10, or 12 weeks. Thus, in some aspects, TMZ in the maintenance dosing regimen can be administered 5 days every 28 days at a dose of 150-200 mg/m² daily for six cycles. In some aspects, TMZ in the maintenance dosing regimen can be administered 5 days during each 28-day cycle for up to 6 chemotherapy cycles. In some aspects, continuation of TMZ treatment beyond 6 cycles (and up to 12 cycle) can be at the discretion of a medical professional.

In some aspects, TMZ, both in the treatment dosing regimen or maintenance dosing regimen is administered orally.

In some aspects, the checkpoint inhibitor is administered once every 1, 2, 3, 4, 5, or 6 weeks. In some aspects, the checkpoint inhibitor is administered once every 3 weeks. For example, in some aspects, the pembrolizumab is administered once every 3 weeks. In some aspects, the checkpoint inhibitor is administered at 100-800 mg per dose. In some aspects, the checkpoint inhibitor is administered at 200 mg per dose. For example, in some aspects, the pembrolizumab is administered at 200 mg per dose. In some aspects, the checkpoint inhibitor is administered intravenously (IV), orally. For example, in some aspects, the pembrolizumab is administered IV. In some aspects, other known checkpoint inhibitors can be administered via their known routes.

In some aspects, the amount of checkpoint inhibitor in the dosing is the same in the treatment phase dosing regimen as it is in the maintenance phase dosing regimen. In some aspects, there is no interruption in dosing with the checkpoint inhibitor between treatment phase and maintenance phase. For example, in some aspects, the maintenance phase dosing regimen can be administered the next day after the last treatment phase dosing regimen, thus there is no break in the every 3 week treatment of checkpoint inhibitor.

In some aspects, the alternating electric field is applied continuously in both the treatment phase dosing regimen and the maintenance phase dosing regimen. In some aspects, the alternating electric field has a frequency and field strength. In some aspects, the alternating electric field has a frequency between 50 kHz and 1 MHz. In some aspects, the alternating electric field has a frequency of about 150 to 250 kHz. In some aspects, the alternating electric field has a frequency of 200 kHz. In some aspects, the alternating electric field has any of the frequencies described throughout. In some aspects, the alternating electric field has a field strength of between 0.5 and 10 V/cm RMS. In some aspects, the alternating electric field has a field strength of about 0.9 V/cm RMS. In some aspects, the alternating electric field has any of the field strengths described throughout.

In some aspects, a dosing regimen comprises the treatment phase dosing regimen and the maintenance phase dosing regimen. For example, in some aspects, a dosing regimen comprises administering radiation therapy (60 Gy in 30 fractions) for 6 weeks; administering a checkpoint inhibitor every 3 weeks (21 days) for up to 35 treatments; applying alternating electric field continuously without interruption until second disease progression or 24 months from enrollment (the earlier of the two); and administering TMZ at a dose of 75mg/m2/day for 6 weeks, then stopping TMZ for 4 to 6 weeks, then administering TMZ at a dose of 150-200 mg/m2 for 5 days during each 28-day cycle for up to 6 chemotherapy cycles.

Disclosed are dosing regimens comprising administering a radiation therapy for 6 weeks; applying, concomitant with the radiation therapy, an alternating electric field to a target site of the subject for six weeks, wherein the target site comprises one or more glioblastoma cells; administering a dose of TMZ at 75mg/m2/day for 6 weeks concomitant with the radiation therapy; and administering a therapeutically effective amount of a checkpoint inhibitor every 3 weeks for 6 weeks concomitant with the radiation therapy, applying, the day after ceasing the 6 weeks of radiation therapy, an alternating electric field to a target site of the subject for a period of time, wherein the target site comprises one or more glioblastoma cells; administering, at least 4 weeks after ceasing the 6 weeks of radiation therapy, a dose of TMZ at 150-200 mg/m2 for 5 days during a 28-day cycle; and administering a continued every 3 week dosing, without interruption from the earlier step of administering a checkpoint inhibitor, of a therapeutically effective amount of a checkpoint inhibitor.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising a checkpoint inhibitor and TMZ. The kits also can comprise equipment (e.g. Optune) for applying alternating electrical fields.

### EXAMPLES

### A. Example 1: A Pilot, Open-Label Study of Optune® Concomitant with Radiation Therapy, Temozolomide, and Pembrolizumab in Newly Diagnosed Glioblastoma

This study investigates the safety profile of administration of TTFields at 200 kHz concurrent with Radiotherapy, TMZ, and Pembrolizumab in newly diagnosed GBM (ndGBM) patients.

### 1. Background

Glioblastoma is an aggressive cancer bearing a very poor prognosis. With the best treatment regimen, which includes maximal debulking surgery, radiation therapy concomitant with temozolomide and maintenance temozolomide concomitant with Optune, the median overall survival is reported to be 20.9 months from the completion of radiation therapy. There is therefore a clear unmet need for additional effective therapies to treat the disease. Despite numerous clinical studies, no therapy has demonstrated improvement in survival of patients during the past several years.

Although immune checkpoint inhibitors improved survival in several other malignancies, they did not yield a clinically significant outcome in multiple clinical trials in glioblastoma. This was accompanied by lack of effector response induction in patients, potentially owing to a scarcity of T cells within the tumor microenvironment. It could therefore be that an induced increase in cytotoxic T cells within the tumor's microenvironment would result in an improvement in the ability of pembrolizumab to augment the immune response against glioblastoma.

Beyond their anti-mitotic effects in glioblastoma and other cancers, TTFields have been reported to preclinically elicit immunogenic cell death, with expression of the hallmark calreticulin and release of the alarmin high-mobility group box 1 (HMGB-1) and adenosine triphosphate (ATP). This, in turn, led to the recruitment of antigen-presenting cells, such as macrophages and dendritic cells, to their activation and finally to the recruitment of cytotoxic T cells into the tumor. Therefore, it could be theorized that in addition to their direct anti-tumor activity, TTFields would augment the immune response by sensitizing the tumor to pembrolizumab: in the presence of cytotoxic T cells accumulated following TTFields application, pembrolizumab would potentially eliminate tumor's resistance to their activity and therefore lead to effective immune response against the tumor. Indeed, this potential synergy between TTFields and pembrolizumab has already been reported clinically through a single arm, phase 2 study. Maintenance temozolomide, Optune and pembrolizumab were used for the treatment of newly diagnosed glioblastoma patients. The analysis showed that PFS, OS and Two-year OS were significantly longer compared to historical controls who received the regimen without pembrolizumab. Molecular analysis prior to the addition of pembrolizumab confirmed robust T cell activation by TTFields. Subsequently, the ability of the top expanded TCRab clones to adapt to the ever-changing tumor microenvironment through successful clonal switching after the addition of pembrolizumab strongly predicted response to the triple combination.

Moreover, TTFields have been demonstrated to increase the therapeutic effects of RT in preclinical models, potentially through effects on the homologous recombination pathway, and inducing a state of ‘BRCAness’ leading to a conditional susceptibility resulting in enhanced sensitivity to ionizing radiation. In two pilot studies TTFields was combined with RT/TMZ, results of these studies demonstrated that TTFields have a high safety profile when combined with radiation therapy in newly diagnosed GBM.

In summary, the current study aims to assess the safety of treatment with TTFields and pembrolizumab during the concomitant chemoradiation treatment in newly diagnosed GBM patients, and to explore the potential for enhancing treatment efficacy.

### 2. Study Design

The current study design is for a Phase I study to look at safety and efficacy of administration of TTFields at 200 kHz concurrent with Radiotherapy, TMZ, and Pembrolizumab in ndGBM patients.

Adult patients with new diagnosis of GBM, of both genders, after surgery or biopsy can be a part of the study. Five subjects will be used for the initial single arm, open label, single-site study.

The duration of the study will be 24 months.

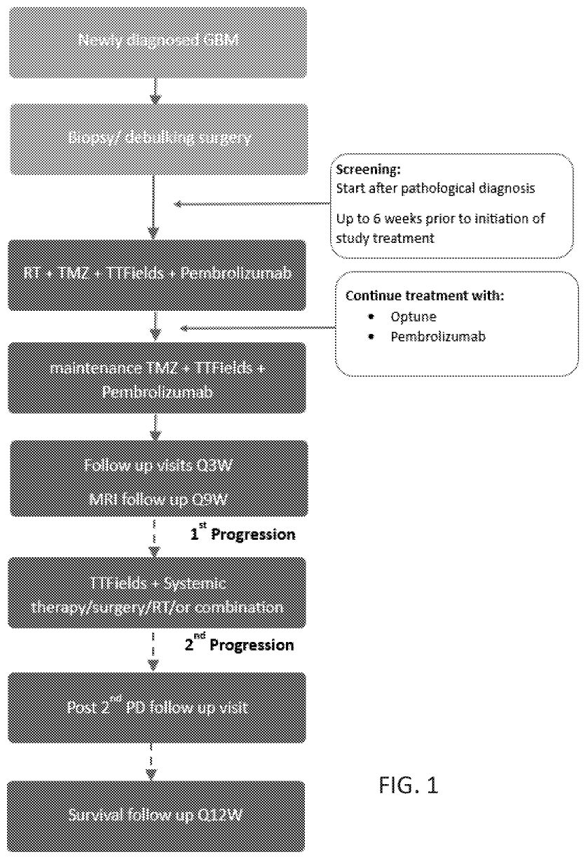

FIG. 1 shows an example of the study schema.

### 3. Objectives

The severity and frequency of adverse events (based on Common Terminology Criteria for Adverse Events, CTCAE V5.0) can be evaluated.

An evaluation of the efficacy of TTFields 200 kHz and pembrolizumab added to standard temozolomide-based radiochemotherapy improving the outcome in ndGBM patients determined by the PFS12 can be performed.

Response rates using (i)RANO (immunotherapy response assessment in neuro-oncology) criteria can be evaluated.

PFS rate at 6 months (PFS6m) and at 12 months (PFS12m) per (i)RANO as can be evaluated.

PFS (PFS2) per mRANO can also be evaluated.

OS using the Kaplan-Meier Analysis (1-year and 2-year survival) can be evaluated.

Time to treatment failure (TTF) can also be evaluated.

The relationship between biomarkers and clinical outcomes following addition of TTFields and pembrolizumab to standard of care treatment (Immunophenotyping, Transcriptional Analyses, Proteomic Analysis, Genomic Analyses) can also be evaluated.

### 4. Inclusion Criteria

The following are the inclusion criteria for subjects of this study:

The participant has provided documented informed consent for the study.

Participants are ≥18 years of age.

Participants can have a new diagnosis of GBM according to WHO 2021 Classification.

Participants can be recovered from maximal debulking surgery (gross total resection, partial resection and biopsy-only patients are all acceptable), Gliadel wafers placement at the time of surgical resection is allowed.

Candidate for standard treatment: Adjuvant chemoradiotherapy of RT according to local practice (56-64 Gy), and concomitant TMZ chemotherapy. Treatment with Optune concomitant with maintenance temozolomide (150-200 mg/m\hat{ }2 daily x5, Q28 days).

All patients must have had tissue submitted for MGMT Promoter Methylation determination prior to start of treatment.

Participants can have an ECOG Performance Status of 0 to 1 assessed within 7 days before start of treatment.

Participants can have a life expectancy ≥3 months.

Participants can have had a maximum dose of 4 mg/day dexamethasone or equivalent doses for other corticosteroids, which has been stable or decreased for ≥5 days prior to start of radiotherapy.

Patients who are treated with anticoagulants are on a stable dose for at least two weeks prior to start of radiotherapy.

The patient can be male or a non-pregnant, non-lactating female.

Females of childbearing potential must have a negative beta-human chorionic gonadotropin (HCG) pregnancy test within 2 weeks prior to receiving the first dose of study medication. Females must practice strict birth control using two different methods (e.g. oral contraceptives in combination with barrier protection) to prevent pregnancy for the duration of the study through 120 days after the last dose of study medication. Males will be advised to use barrier protection starting with the first dose of study therapy through 120 days after the last dose of study therapy).

The patient shows adequate organ functions as assessed by the following laboratory values within 2 weeks prior to first dose of study medication: Participants can have adequate renal function determined by serum creatinine and urea <2 times the upper limit of normal. Participants can have adequate liver function with alanine aminotransferase (ALT), aspartate aminotransferase (AST) and alkaline phosphatase (AP) <3 times the upper limit of normal, and bilirubin value <2.5 mg/dL Participants can have an international normalized ratio (INR) and activated partial thromboplastin time (aPTT) within therapeutic limits (according to the medical standard at the institution). Participants can have hemoglobin >9 g/dL, a platelet count >100×10\hat{ }9/L, a white blood count (WBC) >3×10\hat{ }9/L, and/or an absolute neutrophil count (ANC)>1.5×10\hat{ }9/L.

Participants can be able to have MRI with contrast agent.

### 5. Exclusion Criteria

The following are the exclusion criteria for subjects of this study:

Subjects having infratentorial or leptomeningeal disease.

Subjects having contraindications to the class of drugs under study, e.g. known hypersensitivity or allergy to temozolomide (TMZ) or pembrolizumab.

A subject that is currently participating and receiving study therapy or has participated in a study of an investigational agent and received study therapy or used an investigational device within 4 weeks of the first dose of treatment.

Subjects having a diagnosis of immunodeficiency.

Subjects having human immunodeficiency virus (HIV), hepatitis B virus (HBV) or hepatitis C virus (HCV) infection.

Subjects having a history of active tuberculosis (Bacillus tuberculosis).

Subjects having clinically relevant acute viral, bacterial, or fungal infection.

Subjects having a history of a second independent malignant disorder during the last three years except non-melanoma skin cancer, in situ cervical cancer, treated superficial bladder cancer or cured, early-stage prostate cancer in a patient with prostate-specific antigen (PSA) level less than upper normal limit.

Subjects having an active autoimmune disease that has required systemic treatment in the past 2 years (i.e. with use of disease modifying agents, long-term use of corticosteroids or immunosuppressive drugs). Note: Replacement therapy (eg., thyroxine, insulin, or physiologic corticosteroid replacement therapy for adrenal or pituitary insufficiency, etc.) is not considered a form of systemic treatment.

Subjects having a history of, or any evidence of active, non-infectious pneumonitis.

Subjects having an active infection requiring systemic therapy.

Subjects having a history or current evidence of any condition, therapy, or laboratory abnormality that might confound the results of the trial, interfere with the subject's participation for the full duration of the trial, or is not in the best interest of the subject to participate, in the opinion of the treating investigator.

Subjects having received prior therapy with an anti-programmed cell death (PD)-1, anti-PD-L1, or anti-PD-L2 agent.

Subjects having prior radiotherapy to the brain or interstitial brachytherapy.

Subjects having prior chemotherapy for a brain tumor.

Subjects having intraoperative placement of carmustine wafers (Gliadel®).

Subjects having prior therapy with immune checkpoint inhibitors or vaccination therapy against the tumor.

Subjects having concurrent administration of any antitumor therapy other than standard treatment.

Subjects having clinically relevant psychiatric disorders/legal incapacity or a limited legal capacity

Subjects having received a live vaccine within 30 days of planned start of study therapy. Note: Seasonal influenza vaccines for injection are generally inactivated flu vaccines and are allowed; however intranasal influenza vaccines (e.g., Flu-Mist®) are live attenuated vaccines, and are not allowed.

Women who are pregnant or breast feeding.

Subjects having the intention to become pregnant during the course of the study.

Subjects having a lack of safe contraception, defined as: Female participants of childbearing potential, not using and not willing to continue using a medically reliable method of contraception for the entire study duration, such as oral, injectable, or implantable contraceptives, or intrauterine contraceptive devices, or who are not using any other method considered sufficiently reliable by the investigator in individual cases. Note: female participants who are surgically sterilised/hysterectomised or post-menopausal for longer than 2 years are not considered as being of childbearing potential.

Subjects having the inability to follow the procedures of the study, e.g. due to language problems, psychological disorders, dementia, etc. of the participant.

Subjects having psychiatric or substance abuse disorders including extensive use of alcohol that would interfere with cooperation with the requirements of the trial.

Subjects having had an allogenic tissue/solid organ transplant.

Subjects having early progressive disease before treatment start. If pseudoprogression is suspected, additional imaging studies should be performed to rule out true progression.

Subjects having an implanted pacemaker, defibrillator, deep brain stimulator, other implanted electronic devices in the brain, or documented clinically significant arrhythmias.

Subjects having a skull defect (such as, missing bone with no replacement) or bullet fragments.

Subjects having evidence of increased intracranial pressure (midline shift>5 mm, clinically significant papilledema, vomiting and nausea or reduced level of consciousness.

Subjects having known allergies to medical adhesives or conductive materials.

### B. Example 2

In some aspects, a disclosed treatment plan can be as follows:

Treatment phase:
1. Radiotherapy (60 Gy in 30 fractions) for 6 weeks. Then stop.
2. Pembrolizumab—every 3 weeks (21 days)—this treatment continues every 3 weeks without interruption between the treatment to maintenance phases.
3. TTFields treatment continuously without interruption between the treatment to maintenance phases. TTFields treatment will continue until second disease progression or 24 months from enrollment (the earlier of the two).
4. TMZ-concomitant TMZ at a dose of 75mg/m2/day for 6 weeks->Four to six weeks break.
   Maintenance phase:
1. Pembrolizumab—continue every 3 weeks (21 days). Can be administered for up to 35 treatments.
2. Continue TTFields treatment. TTFields treatment will continue until second disease progression or 24 months from enrollment (the earlier of the two).
3. TMZ—maintenance TMZ will start 4-6 weeks after the end of radiation->TMZ at a dose of 150-200 mg/m2 for 5 days during each 28-day cycle for up to 6 chemotherapy cycles. Continuation of TMZ treatment beyond 6 cycles (and up to 12 cycle) is at the discretion of the treating investigator.

### EMBODIMENTS

Embodiment 1: A method of treating a subject having glioblastoma comprising: applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject.

Embodiment 2: The method of embodiment 1, wherein the checkpoint inhibitor is pembrolizumab (Keytruda), ipilimumab (Yervoy), nivolumab (Opdivo), cemiplimab (trade name Libtayo), and dostarlimab (Jemperli), atezolizumab (Tecentriq), durvalumab (Imfinzi), or avelumab (Bavencio).

Embodiment 3: The method of embodiment 2, wherein the checkpoint inhibitor is pembrolizumab.

Embodiment 4: The method of embodiment 3, wherein the pembrolizumab is administered once every 3 weeks.

Embodiment 5: The method of any one of embodiments 3-4, wherein the pembrolizumab is administered at 200 mg per dose.

Embodiment 6: The method of any one of embodiments 3-5, wherein the pembrolizumab is administered IV.

Embodiment 7: The method of any one of embodiments 1-6, wherein the method of treating is a first line of treatment for glioblastoma.

Embodiment 8: The method of any one of embodiments 1-7, wherein the TMZ is administered daily for six weeks concomitant with the radiation therapy.

Embodiment 9: The method of any one of embodiments 1-8, wherein the TMZ is administered at 75 mg/m² per dose.

Embodiment 10: The method of any one of embodiments 1-9, further comprising halting radiation therapy after four to eight weeks.

Embodiment 11: The method of any one of embodiments 1-9, further comprising halting radiation therapy after six weeks

Embodiment 12: The method of embodiment 10 or 11, further comprising a maintenance TMZ treatment after completion of the radiation therapy.

Embodiment 13: The method of embodiment 12, wherein the maintenance TMZ treatment is administered for five days every 28 days.

Embodiment 14: The method of embodiments 12-13, wherein the maintenance TMZ treatment is administered at 150-200 mg/m² per dose.

Embodiment 15: The method of any one of embodiments 1-14, wherein the TMZ is administered orally.

Embodiment 16: The method of any one of embodiments 1-15, wherein the radiation therapy is administered five days a week for six weeks.

Embodiment 17: The method of embodiment 16, wherein a daily dose of radiation is 2 Gy.

Embodiment 18: The method of any one of embodiments 1-17, wherein a total dose of radiation is 60 Gy.

Embodiment 19: The method of any one of embodiments 1-18, wherein the method of treatment comprises six weeks of radiation therapy and four to twenty-four weeks of alternating electric fields, TMZ, and checkpoint inhibitor.

Embodiment 20: The method of embodiment 19, wherein the six weeks of radiation therapy comprises concomitant treatment with alternating electric fields, TMZ, and checkpoint inhibitor.

Embodiment 21: The method of any one of embodiments 1-20, wherein the alternating electric field has a frequency and field strength.

Embodiment 22: The method of embodiment 21, wherein the alternating electric field has a frequency between 50 kHz and 1 MHz.

Embodiment 23: The method of embodiment 22, wherein the alternating electric field has a frequency of about 150 to 250 kHz.

Embodiment 24: The method of any one of embodiments 21-23, wherein the alternating electric field has a field strength of between 0.5 and 10 V/cm RMS.

Embodiment 25: The method of embodiment 23, wherein the alternating electric field has a field strength of about 0.9 V/cm RMS.

Embodiment 26: The method of any of the preceding embodiments, wherein the subject has been newly diagnosed with glioblastoma (according to WHO 2021 Classification).

Embodiment 27: The method of any of the preceding embodiments, wherein the subject is a candidate for standard chemotherapy and radiation therapy and/or alternating electrical field therapy.

Embodiment 28: The method of any of the preceding embodiments, wherein the subject has undergone surgery.

Embodiment 29: The method of any of the preceding embodiments, wherein the subject has undergone a biopsy.

Embodiment 30: The method of any of the preceding embodiments, wherein the subject has been treated with a corticosteroid for at least 5 days prior to radiation therapy.

Embodiment 31: The method of any of the preceding embodiments, wherein the subject has a life expectancy of at least 3 months.

Embodiment 32: The method of any of the preceding embodiments, wherein the subject does not have an acute viral, bacterial, or fungal infection.

Embodiment 33: The method of any of the preceding embodiments, wherein the subject has not received prior chemotherapy for a brain tumor.

Embodiment 34: The method of any of the preceding embodiments, wherein the subject has not received prior radiation therapy to the brain.

Embodiment 35: The method of any of the preceding embodiments, wherein the subject has not previously treated with TMZ.

Embodiment 36: The method of any of the preceding embodiments, wherein the subject's survival is longer compared to an age-matched subject treated with the standard therapy of concomitant radio-chemotherapy, followed by alternating electric field and maintenance TMZ, without pembrolizumab.

Embodiment 37: The method of any of embodiments 1-34, wherein the subject's survival is longer compared to an age-matched subject treated with standard therapy of chemotherapy and radiation therapy, without TTFields.

Embodiment 38: A dosing regimen comprising: applying an alternating electric field to a target site of the subject for a period of time, wherein the target site comprises one or more glioblastoma cells; administering a radiation therapy; administering a therapeutically effective amount of temozolomide (TMZ); and administering a therapeutically effective amount of a checkpoint inhibitor to the subject, wherein radiation therapy is administered to the subject for 6 weeks.

Embodiment 39: The dosing regimen of embodiment 38, wherein after the six weeks, the method comprises: applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject, wherein no radiation therapy is applied.

Embodiment 40: A dosing regimen comprising:
applying an alternating electric field to a target site of the subject for a period of time, wherein the target site comprises one or more glioblastoma cells; administering a therapeutically effective amount of temozolomide (TMZ); and administering a therapeutically effective amount of a checkpoint inhibitor to the subject, wherein no radiation therapy is applied.

Embodiment 41: The dosing regimen of any one of embodiments 38-39, wherein the TMZ is administered for five days every 28 days concomitant with the radiation therapy.

Embodiment 42: The dosing regimen of any one of embodiments 38-39 and 41, wherein the TMZ is administered at 75 mg/m² per dose.

Embodiment 43: The dosing regimen of embodiment 40, wherein the TMZ administered after the six weeks is administered for five days every 28 days.

Embodiment 44: The dosing regimen of embodiment 40 or 43, wherein the maintenance TMZ treatment is administered at 150-200 mg/m² per dose.

Embodiment 45: The dosing regimen of any one of embodiments 38-444, wherein the checkpoint inhibitor is pembrolizumab.

Embodiment 46: The dosing regimen of embodiment 45, wherein the pembrolizumab is administered once every 3 weeks.

Embodiment 47: The dosing regimen of any one of embodiments 45-46, wherein the pembrolizumab is administered at 200 mg per dose.

Embodiment 48: The dosing regimen of any one of embodiments 44-46, wherein the pembrolizumab is administered IV.

Embodiment 49: The dosing regimen of any one of embodiments 38-48, wherein the alternating electric field has a frequency and field strength.

Embodiment 50: The dosing regimen of embodiment 49, wherein the alternating electric field has a frequency between 50 kHz and 1 MHz.

Embodiment 51: The dosing regimen of embodiment 50, wherein the alternating electric field has a frequency of about 150 to 250 kHz.

Embodiment 52: The dosing regimen of any one of embodiments 49-51, wherein the alternating electric field has a field strength of between 0.5 and 10 V/cm RMS.

Embodiment 53: The method of embodiment 52, wherein the alternating electric field has a field strength of about 0.9 V/cm RMS.

Embodiment 54: A method of increasing survival of a subject having glioblastoma comprising: applying an alternating electric field to a target site of the subject for a period of time, wherein the target site comprises one or more glioblastoma cells; administering a radiation therapy; administering a therapeutically effective amount of temozolomide; and administering a therapeutically effective amount of a checkpoint inhibitor to the subject.

Embodiment 55: The method of embodiment 54, wherein the subject's survival is longer compared to an age-matched subject treated with the standard therapy of concomitant radio-chemotherapy, followed by alternating electric field and maintenance TMZ, without pembrolizumab.

Embodiment 56: The method of embodiment 54, wherein the subject's survival is longer compared to an age-matched subject treated with standard therapy of chemotherapy and radiation therapy, without TTFields.

Embodiment 57: The method of any one of embodiments 54-56, wherein the checkpoint inhibitor is pembrolizumab (Keytruda), ipilimumab (Yervoy), nivolumab (Opdivo), cemiplimab (trade name Libtayo), and dostarlimab (Jemperli), atezolizumab (Tecentriq), durvalumab (Imfinzi), or avelumab (Bavencio).

Embodiment 58: The method of embodiment 57, wherein the checkpoint inhibitor is pembrolizumab.

Embodiment 59: The method of embodiment 58, wherein the pembrolizumab is administered once every 3 weeks.

Embodiment 60: The method of any one of embodiments 58-59, wherein the pembrolizumab is administered at 200 mg per dose.

Embodiment 61: The method of any one of embodiments 58-60, wherein the pembrolizumab is administered IV.

Embodiment 62: The method of any one of embodiments 54-61, wherein the TMZ is administered for five days every 28 days concomitant with the radiation therapy.

Embodiment 63: The method of any one of embodiments 54-62, wherein the TMZ is administered at 75 mg/m² per dose.

Embodiment 64: The method of any one of embodiments 54-63, further comprising halting radiation therapy after four to six weeks.

Embodiment 65: The method of embodiment 64, further comprising a maintenance TMZ treatment upon completion of the radiation therapy.

Embodiment 66: The method of embodiment 65, wherein the maintenance TMZ treatment is administered for five days every 28 days.

Embodiment 67: The method of embodiments 65-66, wherein the maintenance TMZ treatment is administered at 150-200 mg/m² per dose.

Embodiment 68: The method of any one of embodiments 54-67, wherein the TMZ is administered orally.

Embodiment 69: The method of any one of embodiments 54-68, wherein the radiation therapy is administered five days a week for six weeks.

Embodiment 70: The method of embodiment 69, wherein a daily dose of radiation is 2 Gy.

Embodiment 71: The method of any one of embodiments 54-70, wherein a total dose of radiation is 60 Gy.

Embodiment 72: The method of any one of claims 54-71, wherein the method of treatment comprises six weeks of radiation therapy and four to twenty-four weeks of alternating electric fields, TMZ, and checkpoint inhibitor.

Embodiment 73: The method of embodiment 72, wherein the six weeks of radiation therapy comprises concomitant treatment with alternating electric fields, TMZ, and checkpoint inhibitor.

Embodiment 74: The method of any one of embodiments 54-73, wherein the alternating electric field has a frequency and field strength.

Embodiment 75: The method of embodiment 74, wherein the alternating electric field has a frequency between 50 kHz and 1 MHz.

Embodiment 76: The method of embodiment 75, wherein the alternating electric field has a frequency of about 150 to 250 kHz.

Embodiment 77: The method of any one of embodiments 74-76, wherein the alternating electric field has a field strength of between 0.5 and 10 V/cm RMS.

Embodiment 78: The method of embodiment 77, wherein the alternating electric field has a field strength of about 0.9 V/cm RMS.

Embodiment 79: The method of any of the preceding embodiments, wherein the subject has been newly diagnosed with glioblastoma (according to WHO 2021 Classification).

Embodiment 80: The method of any of the preceding embodiments, wherein the subject is a candidate for standard chemotherapy and radiation therapy and/or alternating electrical field therapy.

Embodiment 81: The method of any of the preceding embodiments, wherein the subject has undergone surgery.

Embodiment 82: The method of any of the preceding embodiments, wherein the subject has undergone a biopsy.

Embodiment 83: The method of any of the preceding embodiments, wherein the subject has been treated with a corticosteroid for at least 5 days prior to radiation therapy.

Embodiment 84: The method of any of the preceding embodiments, wherein the subject has a life expectancy of at least 3 months.

Embodiment 85: The method of any of the preceding embodiments, wherein the subject does not have an acute viral, bacterial, or fungal infection.

Embodiment 86: The method of any of the preceding embodiments, wherein the subject has not received prior chemotherapy for a brain tumor.

Embodiment 87: The method of any of the preceding embodiments, wherein the subject has not received prior radiation therapy to the brain.

Embodiment 88: The method of any of the preceding embodiments, wherein the subject has not previously treated with TMZ.

Embodiment 89: A method of enhancing treatment efficacy of an alternating electric field in a subject having glioblastoma comprising: applying an alternating electric field to a target site of the subject for a period of time, wherein the target site comprises one or more glioblastoma cells; administering a radiation therapy; administering a therapeutically effective amount of temozolomide; and administering a therapeutically effective amount of a checkpoint inhibitor to the subject.

Embodiment 90: The method of embodiment 89, wherein the checkpoint inhibitor is pembrolizumab (Keytruda), ipilimumab (Yervoy), nivolumab (Opdivo), cemiplimab (trade name Libtayo), and dostarlimab (Jemperli), atezolizumab (Tecentriq), durvalumab (Imfinzi), or avelumab (Bavencio).

Embodiment 91: The method of embodiment 90, wherein the checkpoint inhibitor is pembrolizumab.

Embodiment 92: The method of embodiment 91, wherein the pembrolizumab is administered once every 3 weeks.

Embodiment 93: The method of any one of embodiments 91-92, wherein the pembrolizumab is administered at 200 mg per dose.

Embodiment 94: The method of any one of embodiments 91-93, wherein the pembrolizumab is administered IV.

Embodiment 95: The method of any one of embodiments 89-94, wherein the method of treating is a first line of treatment for glioblastoma.

Embodiment 96: The method of any one of embodiments 89-95, wherein the TMZ is administered for five days every 28 days concomitant with the radiation therapy.

Embodiment 97: The method of any one of embodiments 89-96, wherein the TMZ is administered at 75 mg/m² per dose.

Embodiment 98: The method of any one of embodiments 89-97, further comprising halting radiation therapy after four to six weeks.

Embodiment 99: The method of embodiment 98, further comprising a maintenance TMZ treatment upon completion of the radiation therapy.

Embodiment 100: The method of embodiment 99, wherein the maintenance TMZ treatment is administered for five days every 28 days.

Embodiment 101: The method of embodiments 99-100, wherein the maintenance TMZ treatment is administered at 150-200 mg/m² per dose.

Embodiment 102: The method of any one of embodiments 99-101, wherein the TMZ is administered orally.

Embodiment 103: The method of any one of embodiments 89-102, wherein the radiation therapy is administered five days a week for six weeks.

Embodiment 104: The method of embodiment 103, wherein a daily dose of radiation is 2 Gy.

Embodiment 105: The method of any one of embodiments 89-104, wherein a total dose of radiation is 60 Gy.

Embodiment 106: The method of any one of embodiments 89-105, wherein the method of treatment comprises six weeks of radiation therapy and four to twenty-four weeks of alternating electric fields, TMZ, and checkpoint inhibitor.

Embodiment 107: The method of embodiment 106, wherein the six weeks of radiation therapy comprises concomitant treatment with alternating electric fields, TMZ, and checkpoint inhibitor.

Embodiment 108: The method of any one of embodiments 89-107, wherein the alternating electric field has a frequency and field strength.

Embodiment 109: The method of embodiment 108, wherein the alternating electric field has a frequency between 50 kHz and 1 MHz.

Embodiment 110: The method of embodiment 109, wherein the alternating electric field has a frequency of about 150 to 250 kHz.

Embodiment 111: The method of any one of embodiments 108-110, wherein the alternating electric field has a field strength of between 0.5 and 10 V/cm RMS.

Embodiment 112: The method of embodiment 111, wherein the alternating electric field has a field strength of about 0.9 V/cm RMS.

Embodiment 113: The method of any of embodiments 89-112, wherein the subject has been newly diagnosed with glioblastoma (according to WHO 2021 Classification).

Embodiment 114: The method of any of embodiments 89-113, wherein the subject is a candidate for standard chemotherapy and radiation therapy and/or alternating electrical field therapy.

Embodiment 115: The method of any of embodiments 89-114, wherein the subject has undergone surgery.

Embodiment 116: The method of any of embodiments 89-115, wherein the subject has undergone a biopsy.

Embodiment 117: The method of any of embodiments 89-116, wherein the subject has been treated with a corticosteroid for at least 5 days prior to radiation therapy.

Embodiment 118: The method of any of embodiments 89-117, wherein the subject has a life expectancy of at least 3 months.

Embodiment 119: The method of any of embodiments 89-118, wherein the subject does not have an acute viral, bacterial, or fungal infection.

Embodiment 120: The method of any of embodiments 89-119, wherein the subject has not received prior chemotherapy for a brain tumor.

Embodiment 121: The method of any of embodiments 89-120, wherein the subject has not received prior radiation therapy to the brain.

Embodiment 122: The method of any of embodiments 89-121, wherein the subject has not previously treated with TMZ.

Embodiment 123: A method of treating a subject having glioblastoma comprising: applying an alternating electric field to a target site of the subject for a period of time, wherein the target site comprises one or more glioblastoma cells; administering a radiation therapy for six weeks; administering a therapeutically effective amount of temozolomide (TMZ) at a dose of 75mg/m2/day during the 6 week radiation therapy, stopping administration of TMZ for at least 4 weeks, and administering TMZ at a dose of 150-200 mg/m2 for five days during a 28 day cycle; administering a therapeutically effective amount of a checkpoint inhibitor every three weeks; wherein applying the alternating electric field continues until second disease progression or 24 months from enrollment; wherein radiation therapy is no longer administered after six weeks.

Embodiment 124: A dosing regimen comprising: administering a radiation therapy for 6 weeks; applying, concomitant with the radiation therapy, an alternating electric field to a target site of the subject for six weeks, wherein the target site comprises one or more glioblastoma cells; administering a dose of TMZ at 75 mg/m2/day for 6 weeks concomitant with the radiation therapy; and administering a therapeutically effective amount of a checkpoint inhibitor every 3 weeks for 6 weeks concomitant with the radiation therapy, applying, the day after ceasing the 6 weeks of radiation therapy, an alternating electric field to a target site of the subject for a period of time, wherein the target site comprises one or more glioblastoma cells; administering, at least 4 weeks after ceasing the 6 weeks of radiation therapy, a dose of TMZ at 150-200 mg/m2 for 5 days during a 28-day cycle; and administering a continued every 3 week dosing, without interruption from step d), of a therapeutically effective amount of a checkpoint inhibitor.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject having glioblastoma comprising:
a. applying an alternating electric field to a target site of the subject for a period of time, wherein the target site comprises one or more glioblastoma cells;
b. administering a radiation therapy;
c. administering a therapeutically effective amount of temozolomide (TMZ); and
d. administering a therapeutically effective amount of a checkpoint inhibitor to the subject.

2. The method of claim 1, wherein the checkpoint inhibitor is pembrolizumab (Keytruda), ipilimumab (Yervoy), nivolumab (Opdivo), cemiplimab (trade name Libtayo), and dostarlimab (Jemperli), atezolizumab (Tecentriq), durvalumab (Imfinzi), or avelumab (Bavencio).

3. The method of claim 2, wherein the pembrolizumab is administered once every 3 weeks.

4. The method of claim 2, wherein the pembrolizumab is administered at 200 mg per dose.

5. The method of claim 1, wherein the TMZ is administered daily for six weeks concomitant with the radiation therapy.

6. The method of claim 1, wherein the TMZ is administered at 75 mg/m² per dose.

7. The method of claim 1, further comprising halting radiation therapy after four to eight weeks.

8. The method of claim 1, further comprising a maintenance TMZ treatment after completion of the radiation therapy.

9. The method of claim 8, wherein the maintenance TMZ treatment is administered for five days every 28 days.

10. The method of claim 8, wherein the maintenance TMZ treatment is administered at 150-200 mg/m² per dose.

11. The method of claim 1, wherein the radiation therapy is administered five days a week for six weeks.

12. The method of claim 1, wherein a daily dose of radiation is 2 Gy.

13. The method of claim 1, wherein a total dose of radiation is 60 Gy.

14. The method of claim 1, wherein the method of treatment comprises six weeks of radiation therapy and four to twenty-four weeks of alternating electric fields, TMZ, and checkpoint inhibitor.

15. The method of claim 14, wherein the six weeks of radiation therapy comprises concomitant treatment with alternating electric fields, TMZ, and checkpoint inhibitor.

16. The method of claim 1, wherein the alternating electric field has a frequency between 50 kHz and 1 MHz.

17. The method of claim 1, wherein the alternating electric field has a field strength of between 0.5 and 10 V/cm RMS.

18. The method of claim 1, wherein the subject does not have an acute viral, bacterial, or fungal infection, has not received prior chemotherapy for a brain tumor, has not received prior radiation therapy to the brain, and/or has not previously treated with TMZ.

19. A method of enhancing treatment efficacy of an alternating electric field in a subject having glioblastoma comprising:
a. applying an alternating electric field to a target site of the subject for a period of time, wherein the target site comprises one or more glioblastoma cells;
b. administering a radiation therapy;
c. administering a therapeutically effective amount of temozolomide; and
d. administering a therapeutically effective amount of a checkpoint inhibitor to the subject.

20. A method of treating a subject having glioblastoma comprising:
a. applying an alternating electric field to a target site of the subject for a period of time, wherein the target site comprises one or more glioblastoma cells;.
b. administering a radiation therapy for six weeks;
c. administering a therapeutically effective amount of temozolomide (TMZ) at a dose of 75 mg/m2/day during the 6 week radiation therapy, stopping administration of TMZ for at least 4 weeks, and administering TMZ at a dose of 150-200 mg/m2 for five days during a 28 day cycle;
d. administering a therapeutically effective amount of a checkpoint inhibitor every three weeks;
wherein applying the alternating electric field continues until second disease progression or 24 months from enrollment;
wherein radiation therapy is no longer administered after six weeks.
